(19) United States
(12) Patent Application Publication
(10) Pub. No.: US 2023/0071863 A1
(43) Pub. Date: Mar. 9, 2023

(54) MINERAL SUNSCREEN SPRAY

(71) Applicant: L'OREAL, Paris (FR)

(72) Inventors: Yixin YANG, Union, NJ (US); Anil SHAH, Hamilton, NJ (US); Hy Si BUI, Piscataway, NJ (US); Brian BODNAR, Manasquan, NJ (US)

(73) Assignee: L'OREAL, Paris (FR)

(21) Appl. No.: 17/462,479

(22) Filed: Aug. 31, 2021

(51) Int. Cl. A61K 8/04; A61K 8/06; A61K 8/25; A61K 8/362; A61K 8/92; A61K 8/81; A61Q 17/04

(52) U.S. Cl. CPC A61K 8/046; A61K 8/064; A61K 8/25; A61K 8/362; A61K 8/922; A61K 8/8129; A61Q 17/04; A61K 2800/20

(57) ABSTRACT

A stable sprayable cosmetic composition that includes at least one mineral UV filter; at least one oil phase rheology modifier; and water; wherein the cosmetic composition is a water-in-oil composition and wherein the viscosity at shear rate of about 1000 (1/s) is lower than about 110 mPa·s. The cosmetic composition can be sprayed without requiring to be mixed with propellant.

### FIELD OF THE INVENTION

The instant disclosure is directed to skin care compositions that provide UV protection, in particular for protecting skin from ultraviolet (“UV”) radiation, and to methods for using the sunscreen compositions to protect keratinous substrates such as skin and hair from UV radiation.

### BACKGROUND OF THE INVENTION

Prolonged exposure to sunlight and UV light in particular causes damage such as sunburn to the skin, serious skin conditions such as skin cancer, and accelerate the appearance of fine lines and wrinkles in a process commonly referred to as photo-aging. Sunscreens can be used to protect against UV damage via inclusion of active ingredients that either absorb, or scatter UV light. The amount of UV protection afforded by a sunscreen composition is related to the amount and type of UV filter(s) contained therein, which may either consist of chemical UV filters, mineral UV filters, or a mixture. It is desirable for formulators of sunscreen compositions to achieve the best UV protection with the lowest amount of UV filters, since UV filters often impart undesirable look and feel characteristics to sunscreen formulations. In particular, mineral UV filters are very desired by consumers, yet impart a significant white color when applied to the skin.

Sunscreen formulations can be administered to the skin through a variety of dosage methods, which include lotions, creams, sticks, and sprays. Sprayable formulations can offer added convenience to consumers when applying sunscreen; however, require a formulation to be able to be forced through a nozzle and dispensed into the air with control of force, spread, and droplet size distribution of spray. Often, the aspect of the sunscreen spray can be modulated effectively by mixing the sunscreen composition with propellant under pressure, which is then forced through a nozzle to afford a spray.

Mixing with propellant is therefore a desired method for producing a viable spray; however, since usage of propellants is often undesirable due to environmental impact of propellants and flammability of propellants, there is great interest to develop sunscreen formulations which are able to yield a spray without being required to first be mixed with propellant. In so-called “Bag-on-Valve” (BOV) products, the sunscreen is often contained separately from the propellant, which is often pressurized air. The propellant then squeezes the bag when an actuator is depressed, which causes the formula without propellant to be forced through a nozzle. A better spray quality can often be obtained when the formula is mixed with propellant first, so in cases where a sunscreen composition will be administered in a spray without being mixed with propellant, it is very important to control specific formulation parameters that allow for the formulation to create a cosmetically-acceptable spray quality.

The inventors of the instant disclosure discovered ways to formulate a sunscreen composition containing mineral UV filters which can afford a good quality of spray without requiring to be mixed with propellant, such as in BOV products.

### BRIEF SUMMARY OF THE INVENTION

In accordance with various embodiments, provided is a method of formulating a stable sprayable mineral sunscreen using one or more rheology modifiers with the correct rheological profile which produces a good quality of spray without requiring the formula to be mixed with propellant, as in a BOV product.

In a representative embodiment, the stable sprayable cosmetic composition comprises:
a. at least one mineral UV filter;
b. at least one oil phase rheology modifier; and
c. water;

wherein the cosmetic composition is a water-in-oil composition and,

wherein the composition has a viscosity lower than about 110 mPa·s at shear rate of about 1000 (1/s).

In some embodiments, the viscosity is lower than about 100 mPa·s at shear rate of about 1000 (1/s). In some embodiments, the viscosity is lower than about 90 mPa·s at shear rate of about 1000 (1/s) is.

In some embodiments, the composition exhibits a yield stress less than about 2 Pa. In various embodiments, the yield stress is less than about 1.8 Pa. In some embodiments, the yield stress is less than about 1.5 Pa.

In some embodiments, the at least one mineral UV filter is selected from titanium dioxide, zinc oxide, and mixtures thereof.

In some embodiments, the at least one mineral UV filter is present from about 5 to about 50 wt. %, based on the total weight of the cosmetic composition.

In one or more embodiments, the cosmetic compositions further comprise one or more organic UV filters.

In one or more embodiments, the at least one oil phase rheology modifier is selected from hydrogenated jojoba oil, disteardimonium hectorite, polyhydroxystearic acid, sorbitol/sebacic acid copolymer behenate, and mixtures thereof.

In one or more embodiments, the at least one oil phase rheology modifier is present from about 0.01 to about 5 wt. %, based on the total weight of the cosmetic composition.

In one or more embodiments, the at least one oil phase rheology modifier is present from about 0.1 to about 3 wt. %, based on the total weight of the cosmetic composition.

In some embodiments, when the composition is sprayed the distribution of droplets is homogenous.

In one or more embodiments, the composition is aerosolized without requiring mixing with propellant. In some embodiments, the viscosity is less than 100 mPa·s at a shear rate of about 1000 (1/s). In some embodiments, the viscosity is from about 20 to about 2000 Pa·s at shear rate of about 0.01 (1/s). In one or more embodiments, the composition is sprayable.

In some embodiments, the stable sprayable water-in-oil cosmetic composition comprises:
a. from about 5 to about 50 wt. % of at least one mineral UV filter;
b. from about 0.1 to about 5 wt. % of at least one oil phase rheology modifier;
c. from about 10 to about 30 wt. % of water; and
   wherein the cosmetic composition is a water-in-oil composition and,

wherein the composition has a viscosity lower than about 100 mPa·s at a shear rate of about 1000 (1/s); wherein all percentages by weight are based on the total weight of the cosmetic composition.

The instant disclosure is also directed to a method for protecting skin from UV radiation comprising applying an effective amount of the sunscreen composition to the skin.

The inventors of the instant disclosure discovered ways to formulate a sunscreen composition containing mineral UV filters which can afford a good quality of spray without requiring to be mixed with propellant, such as in BOV products.

These and other aspects of the invention are set out in the appended claims and described in greater detail in the detailed description of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

It is to be understood that both the foregoing general description and the following detailed description are exemplary and explanatory only and are not restrictive of the disclosure.

### BRIEF DESCRIPTION OF THE DRAWINGS

FIG. 1 is a graph showing the filament breakup time for Inventive and Comparative Examples.

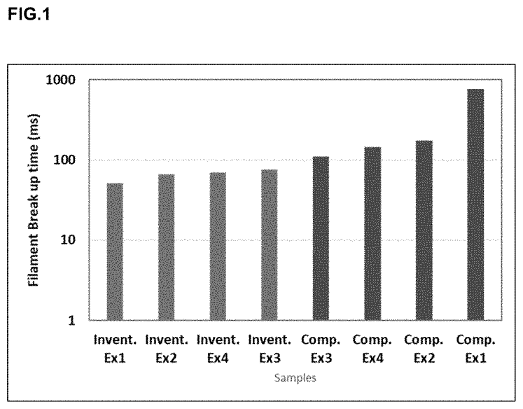

FIG. 2 is a graph showing the yield stress (Pa) for Inventive and Comparative Examples and the correlation between the yield spray and the spray pattern.

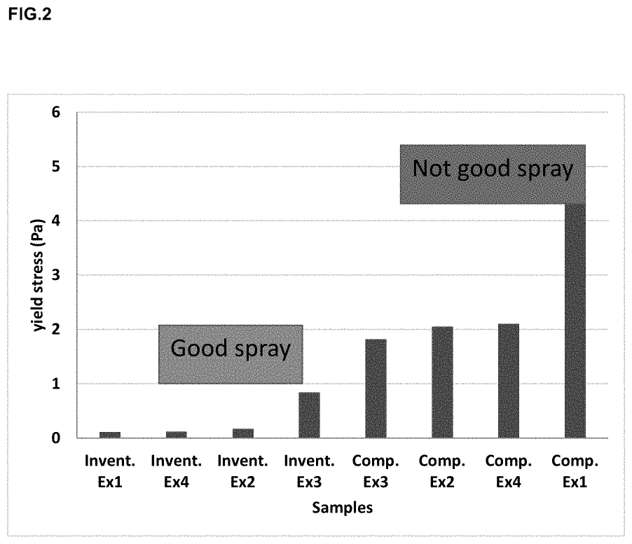

FIG. 3 is a graph showing the correlation between the yield stress (PA) and the filament breakup time (ms). It also shows the correlation between the spray pattern. A good spray pattern is related to a filament breakup time of less than 100 ms.

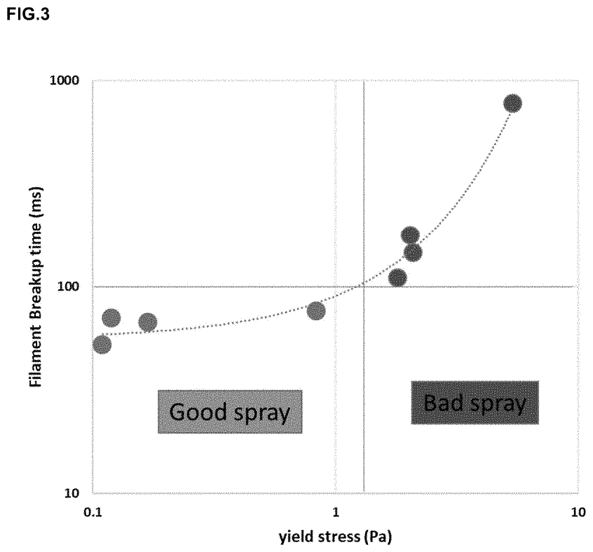

FIG. 4 is a graph showing the shear viscosity (v=1000 1/s) (mPa·S) for Inventive and Comparative Examples. High shear viscosity indicate the sprayable composition with shear viscosity less than about 110 mPa·s

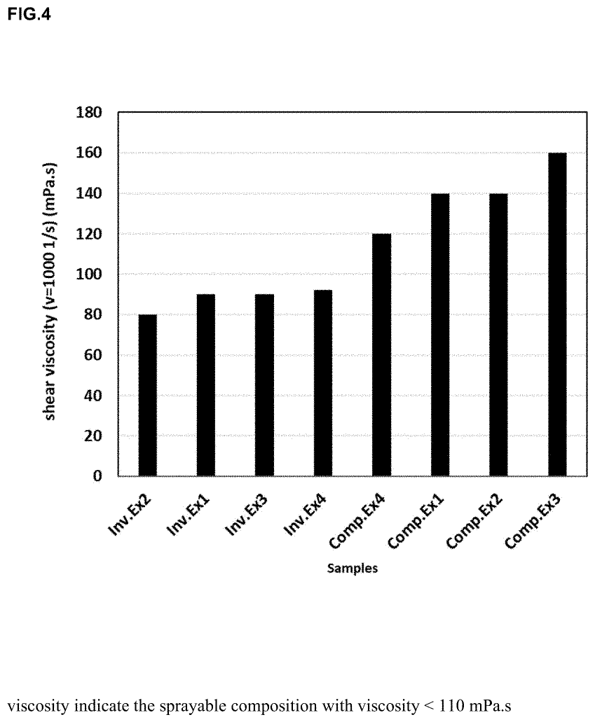

It should be understood that the various aspects of the present disclosure are not limited to the arrangements and instrumentality shown in the figures.

### DETAILED DESCRIPTION OF THE INVENTION

Where the following terms are used in this specification, they are used as defined below.

The term “keratinous tissue” includes, but is not limited to, skin, hair, and nails.

The term “water-in-oil emulsion” or “W/O emulsion” includes a water phase dispersed in an oil phase, where the oil phase is a continuous phase.

The term “Homogenous” means in reference to the composition as having the visual appearance of being substantially uniform throughout, i.e., visually lacks separation and pooling of fluid.

The term “Homogenous” means in reference to the spray pattern as having a distribution of aerosolized droplets which is visually uniform throughout when sprayed in a controlled manner onto a vertical surface.

The term “sun protection factor” or “SPF” is a value expressed mathematically by the ratio of the irradiation time necessary to attain the erythemogenic threshold with the UV screening agent to the time necessary to attain the erythemogenic threshold without UV screening agent. SPF generally provides information about the skin's resistance to ultraviolet B (UVB) radiation from the sun. The SPF rating system has been developed to provide consumer guidance in selecting sunscreens. The sunscreen composition according to the present disclosure can be formulated to achieve a variety of different SPFs. For example, the sunscreen composition can have an in vitro or in vivo SPF of at least 30, 35, 40, 45, 50, or 55 or higher (or in a range between any of these values).

The term “critical wavelength” is an absorption spectrum of a sunscreen composition characterized by an index, namely a wavelength, where the integral of the spectral absorbance curve reached 90% of the integral from 290 nm to 400 nm. The critical wavelength is used to determine the breadth of UV protection. In some embodiments, the sunscreen composition has a critical wavelength that is at least 370 nm.

The terms “water-resistant broad-spectrum UV protection” and “SPF water resistant” and “WR” refer generally to the property of preserved UV protection, for example, stable retention of SPF, after exposure to water under predetermined water exposure times and conditions. A composition demonstrates SPF or UV protection water resistance when the SPF is Suitable water resistance is achieved if a composition demonstrates preserved SPF after being submerged in a base of water after 40 or 80 minutes. Thus, for example, a sunscreen composition may bear the designation WR if the sunscreen demonstrates an SPF that is not diminished by more than 10% after application to a substrate and water submersion for from about 40 minutes up to about 80 minutes.

The term “SPF booster” refers to a material which increases the UV absorption of another material when the two are intermixed in a composition by refracting UV radiation, thereby increasing the effective path length of the UV radiation through the sunscreen composition.

The term “viscosity” refers to the thickness of a fluid or composition and is a measurement of a fluid or composition's resistance to flow. Herein, “viscosity” is synonymous to “dynamic viscosity” or “absolute viscosity”, rather than “kinematic viscosity”, and is measured by means of a rheometer in a method which is known to those skilled-in-the-art. Measurements of viscosity herein are reported in pascal-seconds (Pa·s) or milli pascal-seconds (mPa·s) unless otherwise specified.

The term “sprayable” refers to a sunscreen composition which affords an acceptable quality of spray. Acceptable characteristics of the spray include a narrow droplet size distribution, which may either be a fine mist or consist of larger droplets around about 100 nm. Most typical droplet size distributions for good quality sprays include those with average droplet sizes ranging from about 50 nm to about 200 nm; however, may be larger or smaller depending on the intended application. A good quality spray should also have a spray pattern which is homogenous or mostly homogenous when a proper nozzle is selected for a large portion of the capacity of the spray can. Examples of inhomogenous spray patterns would include streaking and clusters of concentrated droplet distributions, and this may be examined conveniently via visual inspection of spraying the product onto a clean, vertically aligned surface.

The term “INCI” is an abbreviation of International Nomenclature of Cosmetic Ingredients, which is a system of names provided by the International Nomenclature Committee of the Personal Care Products Council to describe personal care ingredients.

The term “weight ratio” or “mass ratio” as used herein, references the amount of a substance in proportion to a mixture containing said substance, and is calculated by dividing the amount of said substance by weight contained in the mixture by the weight of the mixture containing said substance. As an example, a weight ratio of 0.4 for substance A in a mixture of A, B, and C indicates that the weight of substance A divided by the total weight of substances A, B, and C is 0.4.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

Some of the various categories of components identified may overlap. In such cases where overlap may exist and the composition includes both components (or the composition includes more than two components that overlap), an overlapping compound does not represent more than one component. For example, a fatty acid may be characterized as both a nonionic surfactant and a fatty compound. If a particular composition includes both a nonionic surfactant and a fatty compound, a single fatty acid will serve as only the nonionic surfactant or as only the fatty compound (the single fatty acid does not serve as both the nonionic surfactant and the fatty compound).

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

The instant disclosure relates stable sprayable cosmetic compositions that typically include:
a. at least one mineral UV filter;
b. at least one oil phase rheology modifier; and
c. water;

wherein the cosmetic composition is a water-in-oil composition, and wherein the composition has a viscosity at shear rate of 1000 (1/s) lower than about 110 mPa·s.

In some embodiments, the viscosity at shear rate of about 1000 (1/s) is lower than about 100 mPa·s. In some embodiments, the viscosity at shear rate of about 1000 (1/s) is lower than about 90 mPa·s.

In some embodiments, the cosmetic compositions exhibit a viscosity at shear rate of about1000 (1/s) less than about 110 mPa·s, or less than about 100 mPa·s, or less than about 90 mPa·s.

Under extensional condition, the cosmetic compositions exhibit a filament breakup time less than about 100 msec, or less than about 90 msec, or less than about 80 msec.

In some embodiments, the at least one mineral UV filter is selected from titanium dioxide, zinc oxide, iron oxides, cerium oxides, zirconium oxides, and a mixture thereof.

In some embodiments, the at least one mineral UV filter is present from about 5 to about 50 wt. %, based on the total weight of the cosmetic composition.

In one or more embodiments, the cosmetic compositions further comprise one or more organic UV filters.

In one or more embodiments, the cosmetic compositions are applied with an effective amount of the sunscreen compositions to the skin.

UV Filters

In accordance with the various embodiments, the sunscreen composition according to the disclosure includes one or a combination of each of mineral based UV filters and may also include organic UV filters. The organic UV filters are typically present in the oil phase of the emulsion.

Generally, UV filters, active in UV-A and/or UV-B regions, used for the present invention can be water-soluble, fat-soluble or insoluble in commonly used cosmetic solvents. In some exemplary embodiments, the UV filters are selected from oil soluble UV filters. UV-A filters comprise groups of compounds which absorb light predominantly in the range of wavelengths from 320 nm to 400 nm (UV-A), and UV-B filters comprise groups of compounds which absorb light predominantly in the range of wavelengths from 290 nm to 320 nm (UV-B). Examples of suitable UV filters include, may be water-soluble, fat-soluble or insoluble in commonly used cosmetic solvents, and may be inorganic or organic.

Mineral UV Filters

In various embodiments, the sunscreen composition comprises one or a combination of mineral UV filters. The mineral UV filter used for the present disclosure may be active in the UV-A and/or UV-B region. The mineral UV filter may be hydrophilic and/or lipophilic.

In preferred embodiments, the mineral UV filter is in the form of a fine particle such that the mean (primary) particle diameter thereof ranges from 1 nm to 150 nm, and in some embodiments 5 nm to 40 nm, and in some embodiments 10 nm to 30 nm. The mean (primary) particle size or mean (primary) particle diameter herein is an arithmetic mean diameter. In some embodiments, a particle may have a diameter that is has a mean particle size that is about or less than about 1 micron, or that is about or less than about 200 nm, or that is about or less than about 100 nm (sometimes referred to nanoscale). It will be understood by one of ordinary skill in the art that a particle that is referred to as nano scale for purposes of cosmetic applications including for the sunscreen composition according to this disclosure will have a mean particle size that is less than about 100 nm unless specifically identified otherwise, for example as in the case of nano zinc oxide which is sometimes referred to as nanoscale though the UV agent has a mean primary particle size that is less than 200 nm.

In some particular embodiments, the amount of at least one mineral UV filter is present from about 5% to about 50 wt. %, based on the total weight of the sunscreen composition. The total amount of mineral UV filtering agents in the sunscreen compositions can vary but is typically from about 5, 6, 7, 8, 9, 10, 11, 12, 13, 14,15, 16, 17, 18, 19, 20, 21, 22, 23 to about 23, 24, 25, 26, 27, 28, 29, 30, 31, 32, 33, 34, 35, 36, 37, 38, 39, 40, 41, 42, 43, 44, 45, 46, 47, 48, 49 or 50 wt. % based on the total weight of the sunscreen composition.

The mineral UV filter can be selected from the group consisting of metal oxides, which may or may not be coated. And in some embodiments, the mineral UV filters are selected from pigments formed of metal oxides, such as, for example, pigments formed of titanium dioxide (amorphous or crystalline in the rutile and/or anatase form), iron oxide, zinc oxide, zirconium oxide, or cerium oxide, which are all UV photoprotective agents that are well known per se.

The mineral UV filter may or may not be coated, and said coated mineral UV filter may have one or more coating. The coating may comprise one or more compound selected from the group consisting of alumina, silica, aluminum hydroxide, silicones, silanes, fatty acids or salts thereof (such as sodium, potassium, zinc, iron, or aluminum salts), fatty alcohols, lecithin, amino acids, polysaccharides, proteins, alkanolamines, waxes, such as beeswax, (meth)acrylic polymers, organic UV filters, and (per)fluoro compounds. It is in some embodiments desirable for the coating to include one or a combination of organic UV filter.

Of course, the mineral UV filter made of metal oxides may, before their treatment with silicones, have been treated with other surfacing agents, in particular, with cerium oxide, alumina, silica, aluminum compounds, silicon compounds, or their mixtures. The coated mineral UV filter may have been prepared by subjecting the mineral UV filter to one or more surface treatments of a chemical, electronic, mechano-chemical, and/or mechanical nature with any of the compounds as described above, as well as polyethylenes, metal alkoxides (titanium or aluminum alkoxides), metal oxides, sodium hexametaphosphate, and those shown, for example, in Cosmetics & Toiletries, February 1990, Vol. 105, pp. 53-64.

The coated mineral UV filter may be titanium oxides coated: with silica, such as the product “Sun veil” from Ikeda, and “Sunsil TIN 50” from Sunjin Chemical; with silica and with iron oxide, such as the product “Sunveil F” from Ikeda; with silica and with alumina, such as the products “Microtitanium Dioxide MT 500 SA” from Tayca, “Tioveil” from Tioxide, and “Mirasun TiW 60” from Rhodia; with alumina, such as the products “Tipaque TTO-55 (B)” and “Tipaque TTO-55 (A)” from Ishihara, and “UVT 14/4” from Kemira; with alumina and with aluminum stearate, such as the product “Microtitanium Dioxide MT 100 T, MT 100 TX, MT 100 Z or MT-01” from Tayca, the products “Solaveil CT-10 W” and “Solaveil CT 100” from Uniqema, and the product “Eusolex T-AVO” from Merck; with alumina and with aluminum laurate, such as the product “Microtitanium Dioxide MT 100 S” from Tayca; with iron oxide and with iron stearate, such as the product “Microtitanium Dioxide MT 100 F” from Tayca; with zinc oxide and with zinc stearate, such as the product “BR351” from Tayca; with silica and with alumina and treated with a silicone, such as the products “Microtitanium Dioxide MT 600 SAS”, “Microtitanium Dioxide MT 500 SAS”, and “Microtitanium Dioxide MT 100 SAS” from Tayca; with silica, with alumina and with aluminum stearate and treated with a silicone, such as the product “STT-30-DS” from Titan Kogyo; with silica and treated with a silicone, such as the product “UV-Titan X 195” from Kemira; with alumina and treated with a silicone, such as the products “Tipaque TTO-55 (S)” from Ishihara or “UV Titan M 262” from Kemira; with triethanolamine, such as the product “STT-65-S” from Titan Kogyo; with stearic acid, such as the product “Tipaque TTO-55 (C)” from Ishihara; or with sodium hexametaphosphate, such as the product “Microtitanium Dioxide MT 150 W” from Tayca. Other titanium oxide pigments treated with a silicone are, and in some embodiments TiO2 treated with octyltrimethylsilane and for which the mean size of the individual particles is from 25 and 40 nm, such as that marketed under the trademark “T 805” by Degussa Silices, TiO2 treated with a polydimethylsiloxane and for which the mean size of the individual particles is 21 nm, such as that marketed under the trademark “70250 Cardre UF TiO2Si3” by Cardre, and anatase/rutile TiO2 treated with a polydimethylhydrosiloxane and for which the mean size of the individual particles is 25 nm, such as that marketed under the trademark “Microtitanium Dioxide USP Grade Hydrophobic” by Color Techniques.

And in some embodiments, the following coated TiO2 can be used as the coated mineral UV filter: Stearic acid (and) Aluminum Hydroxide (and) TiO2, such as the product “MT-100 TV” from Tayca, with a mean primary particle diameter of 15 nm; Dimethicone (and) Stearic Acid (and) Aluminum Hydroxide (and) TiO2, such as the product “S A-TTO-S4” from Miyoshi Kasei, with a mean primary particle diameter of 15 nm; Silica (and) TiO2, such as the product “MT-100 WP” from Tayca, with a mean primary particle diameter of 15 nm; Dimethicone (and) Silica (and) Aluminum Hydroxide (and) TiO2, such as the product “MT-Y02” and “MT-Y-110 M3S” from Tayca, with a mean primary particle diameter of 10 nm; Dimethicone (and) Aluminum Hydroxide (and) TiO2, such as the product “SA-TTO-S3” from Miyoshi Kasei, with a mean primary particle diameter of 15 nm; Dimethicone (and) Alumina (and) TiO2, such as the product “UV TITAN MI 70” from Sachtleben, with a mean primary particle diameter of 15 nm; and Silica (and) Aluminum Hydroxide (and) Alginic Acid (and) TiO2, such as the product “MT-100 AQ” from Tayca, with a mean primary particle diameter of 15 nm. In terms of UV filtering ability, TiO2 coated with one or a combination of organic UV filter is more desirable. For example, Avobenzone (and) Stearic Acid (and) Aluminum Hydroxide (and) TiO2, such as the product “HXMT-100ZA” from Tayca, with a mean primary particle diameter of 15 nm, can be used.

The uncoated titanium oxide pigments are, for example, marketed by Tayca under the trademarks “Microtitanium Dioxide MT500B” or “Microtitanium Dioxide MT600B”, by Degussa under the trademark “P 25”, by Wacker under the trademark “Oxyde de titane transparent PW”, by Miyoshi Kasei under the trademark “UFTR”, by Tomen under the trademark “ITS” and by Tioxide under the trademark “Tioveil AQ”. The uncoated zinc oxide pigments are, for example: those marketed under the trademark “Z-cote” by Sunsmart; those marketed under the trademark “Nanox” by Elementis; and those marketed under the trademark “Nanogard WCD 2025” by Nanophase Technologies. The coated zinc oxide pigments are, for example: those marketed under the trademark “Oxide Zinc CS-5” by Toshiba (ZnO coated with polymethylhydrosiloxane); those marketed under the trademark “Nanogard Zinc Oxide FN” by Nanophase Technologies (as a 40% dispersion in Finsolv TN, C12-C15 alkyl benzoate); those marketed under the trademark “Daitopersion Zn-30” and “Daitopersion Zn-50” by Daito (dispersions in oxyethylenated polydimethylsiloxane/cyclopolymethylsiloxane comprising 30% or 50% of zinc nano-oxides coated with silica and polymethylhydrosiloxane); those marketed under the trademark “NFD Ultrafine ZnO” by Daikin (ZnO coated with phosphate of perfluoroalkyl and a copolymer based on perfluoroalkylethyl as a dispersion in cyclopentasiloxane); those marketed under the trademark “SPD-Z1” by Shin-Etsu (ZnO coated with a silicone-grafted acrylic polymer dispersed in cyclodimethylsiloxane); those marketed under the trademark “Escalol Z100” by ISP (alumina-treated ZnO dispersed in an ethylhexyl methoxycinnamate/PVP-hexadecene copolymer/methicone mixture); those marketed under the trademark “Fuji ZnO-SMS-10” by Fuji Pigment (ZnO coated with silica and polymethylsilsesquioxane); and those marketed under the trademark “Nanox Gel TN” by Elementis (ZnO dispersed at 55% in C12-C15 alkyl benzoate with hydroxystearic acid polycondensate). The uncoated cerium oxide pigments are marketed, for example, under the trademark “Colloidal Cerium Oxide” by Rhone-Poulenc.

The uncoated iron oxide pigments are, for example, marketed by Arnaud under the trademarks “Nanogard WCD 2002 (FE 45B)”, “Nanogard Iron FE 45 BL AQ”, “Nanogard FE 45R AQ”, and “Nanogard WCD 2006 (FE 45R)”, or by Mitsubishi under the trademark “TY-220”. The coated iron oxide pigments are, for example, marketed by Arnaud under the trademarks “Nanogard WCD 2008 (FE 45B FN)”, “Nanogard WCD 2009 (FE 45B 556)”, “Nanogard FE 45 BL 345”, and “Nanogard FE 45 BL”, or by BASF under the trademark “Oxyde de fer transparent”.

Organic UV Filters

In various embodiments, the sunscreen composition may also comprise one or a combination of organic UV filters. The organic UV filters may be active in the UV-A and/or UV-B region. The organic UV filter may be hydrophilic and/or lipophilic. The organic UV filter may be solid or liquid. The terms “solid” and “liquid” mean solid and liquid, respectively, at 25° C. under 1 atm.

The organic UV filter can be selected from the group consisting of anthranilic compounds; dibenzoylmethane compounds; cinnamic compounds; salicylic compounds; camphor compounds; benzophenone compounds; ÿ,ÿ-diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; screening polymers and screening silicones; dimers derived from a-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; phenols; retinol; cysteine; aromatic amino acids; peptides having an aromatic amino acid residue; and mixtures thereof.

Mention may be made, as examples of the organic UV filter(s), of those denoted below under their INCI names, and mixtures thereof. Anthranilic compounds: Menthyl anthranilate, marketed under the trademark “Neo Heliopan MA” by Haarmann and Reimer. Dibenzoylmethane compounds: Butyl methoxydibenzoylmethane, marketed, in particular, under the trademark “Parsol 1789” by Hoffmann-La Roche; and isopropyl dibenzoylmethane. Cinnamic compounds: Ethylhexyl methoxycinnamate, marketed, in particular, under the trademark “Parsol MCX” by Hoffmann-La Roche; isopropyl methoxycinnamate; isopropoxy methoxycinnamate; isoamyl methoxycinnamate, marketed under the trademark “Neo Heliopan E 1000” by Haarmann and Reimer; cinoxate (2-ethoxyethyl-4-methoxy cinnamate); DEA methoxycinnamate; diisopropyl methylcinnamate; and glyceryl ethylhexanoate dimethoxycinnamate. Salicylic compounds: Homosalate (homomentyl salicylate), marketed under the trademark “Eusolex HMS” by Rona/EM Industries; ethylhexyl salicylate, marketed under the trademark “Neo Heliopan OS” by Haarmann and Reimer; glycol salicylate; butyloctyl salicylate; phenyl salicylate; dipropyleneglycol salicylate, marketed under the trademark “Dipsal” by Scher; and TEA salicylate, marketed under the trademark “Neo Heliopan TS” by Haarmann and Reimer. Camphor compounds, in particular, benzylidenecamphor derivatives: 3-benzylidene camphor, manufactured under the trademark “Mexoryl SD” by Chimex; 4-methylbenzylidene camphor, marketed under the trademark “Eusolex 6300” by Merck; benzylidene camphor sulfonic acid, manufactured under the trademark “Mexoryl SL” by Chimex; camphor benzalkonium methosulfate, manufactured under the trademark “Mexoryl SO” by Chimex; terephthalylidene dicamphor sulfonic acid, manufactured under the trademark “Mexoryl SX” by Chimex; and polyacrylamidomethyl benzylidene camphor, manufactured under the trademark “Mexoryl SW” by Chimex. Benzophenone compounds: Benzophenone-1 (2,4-dihydroxybenzophenone), marketed under the trademark “Uvinul 400” by BASF; benzophenone-2 (Tetrahydroxybenzophenone), marketed under the trademark “Uvinul D50” by BASF; Benzophenone-3 (2-hydroxy-4-methoxybenzophenone) or oxybenzone, marketed under the trademark “Uvinul M40” by BASF; benzophenone-4 (hydroxymethoxy benzophonene sulfonic acid), marketed under the trademark “Uvinul MS40” by BASF; benzophenone-5 (Sodium hydroxymethoxy benzophenone Sulfonate); benzophenone-6 (dihydroxy dimethoxy benzophenone); marketed under the trademark “Helisorb 11” by Norquay; benzophenone-8, marketed under the trademark “Spectra-Sorb UV-24” by American Cyanamid; benzophenone-9 (Disodium dihydroxy dimethoxy benzophenonedisulfonate), marketed under the trademark “Uvinul DS-49” by BASF; and benzophenone-12, and n-hexyl 2-(4-diethylamino-2-hydroxybenzoyl)benzoate (UVINUL A+ by BASF). ÿ,ÿ-Diphenylacrylate compounds: Octocrylene, marketed in particular under the trademark “Uvinul N539” by BASF; and Etocrylene, marketed in particular under the trademark “Uvinul N35” by BASF. Triazine compounds: Diethylhexyl butamido triazone, marketed under the trademark “Uvasorb HEB” by Sigma 3V; 2,4,6-tris(dineopentyl 4′-aminobenzalmalonate)-s-triazine, bis-ethylhexyloxyphenol methoxyphenyl triazine marketed under the trademark «TINOSORB S» by CIBA GEIGY, and ethylhexyl triazone marketed under the trademark «UVTNUL T150» by BASF. Benzotriazole compounds, in particular, phenylbenzotriazole derivatives: 2-(2H-benzotriazole-2-yl)-6-dodecyl-4-methylpheno, branched and linear; and those described in U.S. Pat. No. 5,240,975. Benzalmalonate compounds: Dineopentyl 4′-methoxybenzalmalonate, and polyorganosiloxane comprising benzalmalonate functional groups, such as polysilicone-15, marketed under the trademark “Parsol SLX” by Hoffmann-LaRoche. Benzimidazole compounds, in particular, phenylbenzimidazole derivatives: Phenylbenzimidazole sulfonic acid, marketed in particular under the trademark “Eusolex 232” by Merck, and disodium phenyl dibenzimidazole tetrasulfonate, marketed under the trademark “Neo Heliopan AP” by Haarmann and Reimer. Imidazoline compounds: Ethylhexyl dimethoxybenzylidene dioxoimidazoline propionate. Bis-benzoazolyl compounds: The derivatives as described in EP-669,323 and U.S. Pat. No. 2,463,264. Para-aminobenzoic acid compounds: PABA (p-aminobenzoic acid), ethyl PABA, Ethyl dihydroxypropyl PABA, pentyl dimethyl PABA, ethylhexyl dimethyl PABA, marketed, in particular, under the trademark “Escalol 507” by ISP, glyceryl PABA, and PEG-25 PABA, marketed under the trademark “Uvinul P25” by BASF. Methylene bis-(hydroxyphenylbenzotriazol) compounds, such as 2,2′-methylenebis[6-(2H-benzotriazol-2-yl)-4-methyl-phenol] marketed in the solid form under the trademark “Mixxim BB/200” by Fairmount Chemical, 2,2′-methylenebis[6-(2H-benzotriazol-2-yl)-4-(I,I,3,3-tetramethylbutyl)phenol] marketed in the micronized form in aqueous dispersion under the trademark “Tinosorb M” by BASF, or under the trademark “Mixxim BB/100” by Fairmount Chemical, and the derivatives as described in U.S. Pat. Nos. 5,237,071 and 5,166,355, GB-2,303,549, DE-197,26,184, and EP-893,119, and Drometrizole trisiloxane, marketed under the trademark “Silatrizole” by Rhodia Chimie or- “Mexoryl XL” by L'Oreal. Benzoxazole compounds: 2,4-bis[5-I(dimethylpropyl)benzoxazol-2-yl-(4-phenyl)imino]-6-(2-ethylhexyl)imino-I,3,5-triazine, marketed under the trademark of Uvasorb K2A by Sigma 3V. Screening polymers and screening silicones: The silicones described in WO 93/04665. Dimers derived from a-alkylstyrene: The dimers described in DE-19855649. 4,4-Diarylbutadiene compounds: I,I-dicarboxy(2,2′-dimethylpropyl)-4,4-diphenylbutadiene.

In some embodiments the organic UV filter(s) may be selected from the group consisting of: butyl methoxydibenzoylmethane, ethylhexyl methoxycinnamate, homosalate, ethylhexyl salicylate, phenylbenzimidazole sulfonic acid, benzophenone-3, benzophenone-4, benzophenone-5, n-hexyl 2-(4-diethylamino-2-hydroxybenzoyl)benzoate, I,r-(I,4-piperazinediyl)bis[I-[2-[4-(diethylamino)-2-hydroxybenzoyl]phenyl]-methanone 4-methylbenzylidene camphor, terephthalylidene dicamphor sulfonic acid, disodium phenyl dibenzimidazole tetrasulfonate, ethylhexyl triazone, bis-ethylhexyloxyphenol methoxyphenyl triazine, diethylhexyl butamido triazone, 2,4,6-tris(dineopentyl 4′-aminobenzalmalonate)-s-triazine, 2,4,6-tris(diisobutyl 4′-aminobenzalmalonate)-s-triazine, 2,4-bis-(n-butyl 4′-aminobenzalmalonate)-6-[(3-{1,3,3,3-tetramethyl-1-[(trimethylsilyloxy]- disiloxanyl}propyl)amino]-s-triazine, 2,4,6-tris-(di-phenyl)-triazine, 2,4,6-tris-(ter-phenyl)-triazine, methylene bis-benzotriazolyl tetramethylbutylphenol, drometrizole trisiloxane, polysilicone-15, dineopentyl 4′-methoxybenzalmalonate, I,I-dicarboxy(2,2′-dimethylpropyl)-4,4-diphenylbutadiene, 2,4-bis[5-I(dimethylpropyl)benzoxazol-2-yl-(4-phenyl)imino]-6-(2-ethylhexyl)imino- I,3,5-triazine, camphor benzylkonium methosulfate, and mixtures thereof.

Oil Phase Theology Modifiers

The term “oil phase rheology modifiers” refers to functional ingredients which have an influence on the viscosity of the oil phase. Said oil phase rheology modifiers may also be referred to as “thickeners”, “oil thickeners”, “structuring agent”, or “dispersing agents”, and may comprise but are not limited to polymers, waxes, fatty alcohols and derivatives thereof, inorganic solids, esters, polysaccharides, oils, and clays.

In some embodiments, the sunscreen compositions may include at least one oil phase rheology modifier selected from hydrogenated jojoba oil, disteardimonium hectorite, polyhydroxystearic acid, and sorbitol/sebacic acid copolymer behenate, and mixtures thereof. In one or more embodiments, the at least one oil phase rheology modifier is present from about 0.01 to about 15 wt. %, based on the total weight of the cosmetic composition. In one or more embodiments, the at least one oil phase rheology modifier is present from about 0.1 to about 5 wt. %, based on the total weight of the cosmetic composition.

In one or more embodiments, the composition is sprayable as an aerosol without requiring mixing with propellant. In some embodiments, the viscosity is less than 100 mPa·s at a high shear rate of 1000 s\hat{ }−1. In some embodiments, the viscosity is from about 20 to about 2000 Pa·s at shear rate of 0.01 s\hat{ }−1. In one or more embodiments, the composition is sprayable.

The term “oil phase rheology modifiers” means the provision of effects ranging from increasing the viscosity (viscosity-enhancing or thickening) up to gelation of the oil (creation of a three-dimensional structure at the molecular level which “traps” the continuous oily phase) and includes the possibility of generating phases of liquid crystal type in the oil, all these factors being able to improve the stability of the phases dispersed in the oil.

According to preferred embodiments of the instant disclosure, the compositions may comprise at least one non-wax oil phase rheology modifier.

Preferably, the non-wax oil phase rheology modifier is a naturally-derived substance which has the ability to create a gel structure and/or thicken the composition. Preferably, the non-wax oil phase rheology modifier is suitable for use as a substitute for montan-wax. Preferably, the non-wax oil phase rheology modifier is a copolymer comprising at least one sugar or sugar alcohol such as, for example, sorbitol or mannitol; at least one fatty dicarboxylic acid, wherein the fatty portion contains at least 8 carbon atoms; and at least one fatty acid (as defined above).

Suitable examples of such oil phase rheology modifiers include, but are not limited to, Syncrowax™ OSW and Syncrowax™ ORM available from Croda. Syncrowax™ ORM has the INCI name sorbitol/sebacic acid copolymer behenate. Syncrowax™ OSW is a blend of Tribehenin and Syncrowax™ ORM.

Preferably, the oil phase rheology modifier is present in the compositions of the instant disclosure in amounts of active material generally ranging from about 0.075% to about 15%, preferably from about 0.1% to about 7.5%, preferably from about 0.2% to about 5%, and preferably from about 0.5% to about 2.5%, by weight, based on the total weight of the composition, including all ranges and subranges in between.

In some embodiments, oil phase rheology modifiers that may be used in the sunscreen composition are selected from polyester homopolymers and Modified Clay, Silica or Organic Modified Hectorite Branched-Chain Aliphatic Hydrocarbons. In some particular embodiments, the one or a combination of oil phase rheology modifiers comprises one or more of Polyhydroxystearic Acid and Disteardimonium Hectorite.

In some particular embodiments, one or more of the Polyhydroxystearic Acid is present from about 0.1% to about 2%, and Disteardimonium Hectorite is present from about 0.1% to about 1%, each one or more present by weight, based on the total weight of the sunscreen composition.

Each one or a combination of oil phase rheology modifiers is present in the sunscreen composition at a concentration from about 0.01% to 15%, in some embodiments from about 0.1% to 10%, and in some embodiments from about 0.5% to 5% by weight, all weights based on the total weight of the sunscreen composition. Thus, in various embodiments, an oil phase rheology modifier is present in a composition in a weight percent amount from about 0.01, 0.02, 0.03, 0.04, 0.05, 0.06, 0.07, 0.08, 0.09, 1.0, 2.0, 3.0, 4.0, 5.0, 6.0, 7.0, 8.0, 9.0, 10.0, 11.0, 12.0, 13.0, 14.0, to about 15.0 wt. %, including increments there between.

Emulsifiers

In accordance with the various embodiments, the sunscreen composition according to the instant disclosure includes one or a combination of emulsifiers.

In some embodiments, emulsifiers that may be used in the sunscreen composition are selected from double fatty chain polyester copolymers and polyglyceryl fatty acid glycerol esters. In some embodiments, emulsifiers may be selected from Alkylated Sorbitol Esters and Fatty Ester; Alkylated Fatty Acid Glycol Esters and Fatty Esters; Single Fatty Chain Phosphoric Acid; Sorbitol Esters; Glycerol Ester Triglycerides; and Single Fatty Chain Fatty Acids.

In some particular embodiments, the one or a combination of emulsifiers comprises a double fatty chain polyester copolymer comprising PEG-30 Dipolyhydroxystearate, a polyglyceryl fatty acid glycerol ester comprising Polyglyceryl-4 Isostearate, or a combination thereof, each present from about 2% to about 5%, by weight, based on the total weight of the sunscreen composition.

Each one of the one or a combination of emulsifiers is present in the sunscreen composition at a concentration from about 0.01% to 15%, in some embodiments from about 0.1% to 10%, and in some embodiments from about 0.5% to 5% by weight, all weights based on the total weight of the sunscreen composition. Thus, in various embodiments, an emulsifier is present in a composition in a weight percent amount from about 0.01, 0.02, 0.03, 0.04, 0.05, 0.06, 0.07, 0.08, 0.09, 1.0, 2.0, 3.0, 4.0, 5.0, 6.0, 7.0, 8.0, 9.0, 10.0, 11.0, 12.0, 13.0, 14.0, to about 15.0 wt. %, including increments there between.

Solvents

In accordance with various embodiments, the sunscreen composition according to the instant disclosure includes one or more solvents, including at least one solvent in one or both of the aqueous phase and the oil phase of the emulsion. In some embodiments, the sunscreen composition includes solvents selected from water, propanediol, ethylhexylglycerin, caprylyl glycol, C12-15 alkyl benzoate, dimethicone, caprylyl methicone, dicaprylyl carbonate, cetearyl alcohol, isohexadecane, butyloctyl salicylate, cetearyl alcohol (and) behentrimonium methosulfate, Dicaprylyl Ether, Isononyl Isononanoate, Diisopropyl Sebacate, Isododecane, Isopropyl Myristate, and combinations thereof.

More generally, in various embodiments, the solvents can include one or a combination of water, glycols, and oils/emollients that can include silicone and non-silicone compounds. In the various embodiments, the solvents can be present from about 0.05% to about 60%, or from about 0.05% to about 50%, or from about 0.1% to about 45%, or from about 1% to about 20%, or from about 1% to about 10%, or from about 1% to about 5%, or from about 2% to about 15%, or from about 3% to about 12%, or from about 4% to about 10%, or any suitable combination, sub-combination, range, or sub-range thereof by weight, based on the total weight of the sunscreen composition. One of ordinary skill in the art, however, will appreciate that other ranges are within the scope of the invention.

In some particular embodiments, the solvents can include water present in an amount that is from about 10% to about 45%. In some particular embodiments, solvents can include dimethicone and caprylyl methicone, each present from about 4% to about 10% by weight, based on the total weight of the sunscreen composition.

Thus, any one solvent or combination of solvents may be present, by weight, based on the total weight of the sunscreen composition, is from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 55 to about 60 weight percent, including increments and ranges therein and there between.

Powders

In accordance with some embodiments, the sunscreen composition according to the disclosure may comprise one or a combination of sensorial feel modifying powders. In some particular embodiments, the powders may be selected from boron nitride, perlite, and aluminium starch octenyl succinate.

The one or combination of sensorial feel modifying powders, when present, may be at a concentration from about 0.05% to 15%, in some embodiments from about 0.1% to 10%, and in some embodiments from about 1% to 5% by weight, all weights based on the total weight of the sunscreen composition. Thus, in various embodiments, a powder, when present, may be present in a composition in a weight percent amount from 0.05, 0.06, 0.07, 0.08, 0.09, 1.0, 2.0, 3.0, 4.0, 5.0, 6.0, 7.0, 8.0, 9.0, 10.0, 11.0, 12.0, 13.0, 14.0, to about 15.0 percent by weight, including increments there between.

Hydrating Agents

In accordance with the disclosure, in some embodiments, one or a combination hydrating agents or humectants may be present in the sunscreen composition. The hydrating agent present in the cosmetic composition, according to the disclosure, includes, but is not limited to, one or a combination of polyols, including, for example, glycerin, glycerol, glycols, such as caprylyl glycol, butylene glycol, propylene glycol, isoprene glycol, dipropylene glycol, hexylene glycol and polyethylene glycols, monoethylene glycol, diethylene glycol, triethylene glycol, diethylene glycol, hexylene glycol, glycol ethers such as monopropylene, dipropylene and tripropylene glycol alkyl(C1-C4)ethers, squalane, triacetin, sugars, such as glucose, xylitol, maltitol, sorbitol, sucrose pentaerythritol, inositol, pyrrolidone carboxylic acid, lactic acid, lithium chloride, acetamide MEA, sodium lactate, urea, dicyanamide, hyaluronic acid, aloe vera, honey, and seaweed extract.

In some embodiments, the sunscreen composition includes a hydrating agent selected from one or a combination of glycerin present at about 4% and caprylyl glycol present at about 1%.

In accordance with the various embodiments, the amount of hydrating agent present in the sunscreen composition can range from about 1% to about 25%, or from about 2% to about 20%, or from about 3% to about 5% or any suitable combination, sub-combination, range, or sub-range thereof by weight, based on the total weight of the sunscreen composition. One of ordinary skill in the art, however, will appreciate that other ranges are within the scope of the invention.

Thus, any one of or a combination of hydrating agent may be present, by weight, based on the total weight of the sunscreen composition, is from about 1, 2, 3, 4, 5, 6, 7, 8, 9, 10, 11, 12, 13, 14, 15, 16, 17, 18, 19, 20, 21, 22, 23, 24, to about 25 weight percent, including increments and ranges therein and there between.

Actives

The sunscreen compositions can also comprise one or a combination of actives used in the cosmetics field which does not affect the properties of the sunscreen compositions according to the invention, such as fragrances, pearlescent agents, silica, preservatives, proteins, protein hydrolysates, sodium hyaluronate, vitamins, panthenol, silicones, odor absorbers and coloring materials; anti-microbial components, including, but not limited to, phenoxyethanol, chlorphenesin, capryloyl glycol and sodium salicylate; essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang; fruit extracts, for example Pyrus Malus (Apple) Fruit Extract, and Aloe Barbadensis Leaf Juice Powder; citric acid, sodium chloride; neutralizing or pH-adjusting agents (e.g., triethylamine (TEA) and sodium hydroxide), and combinations thereof.

Although the optional actives are given as examples, it will be appreciated that other optional components compatible with cosmetic applications known in the art may be used. In some particular embodiments, the sunscreen composition may include antimicrobials comprising one or a combination of capryloyl glycol and phenoxyethanol.

In some particular embodiments, the sunscreen composition may include actives comprising one or a combination of Disodium EDTA, Sodium Hyaluronate, Sodium Lauroyl Lactylate (And) Ceramide NP (And) Ceramide AP (And) Phytosphingosine (And) Cholesterol (And) Xanthan Gum (And) Carbomer (And) Ceram ide EOP, Trisodium Ethylenediamine Disuccinate, panthenol, tocopherol, niacinamide, and Magnesium Sulfate.

In accordance with the various embodiments, the amount of each one or a combination of actives, when present in the sunscreen composition can be present in a range from about 0.001% to about 20%, by weight, or from about 0.005% to about 0.01%, or from about 0.01% to about 0.1%, or from about 0.15% to about 5%, or from about 0.40% to about 4%, or from about 0.5% to about 2.5% by weight, or from about 1% to about 2%, or any suitable combination, sub-combination, range, or sub-range thereof by weight, based on the total weight of the sunscreen composition. And in some embodiments, a combination of actives present in the sunscreen composition can be present in a range from about 0.001% to about 20%.

Thus, any one or a combination of actives, when present, may be present, by weight, based on the total weight of the sunscreen composition, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 weight percent, including increments and ranges therein and there between.

More exhaustive but non-limiting lists of components useful in the hair care compositions disclosed herein are presented below.

Implementation of the present disclosure is provided by way of the following examples. The examples serve to illustrate the technology without being limiting in nature.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

### EXAMPLES

Implementation of the present disclosure is provided by way of the following examples. The following examples serve to elucidate aspects of the technology without being limiting in nature.

### Example 1

### Inventive and Comparative Compositions

TABLE 1
Inventive and Comparative Compositions

| | Inventive | Inventive | Inventive | Inventive | Comparative | Comparative | Comparative | Comparative |
|---|---|---|---|---|---|---|---|---|
| INCI US | Ex. 1 | Ex. 2 | Ex. 3 | Ex. 4 | Ex. 1 | Ex. 2 | Ex. 3 | Ex. 4 |
| Zinc Oxide | 14.2 | 12.5 | 12.5 | 12.5 | 12.5 | 12.5 | 12.5 | 10.0 |
| Titanium Dioxide | 5.8 | 8.9 | 8.9 | 10.0 | 7.5 | 8.5 | 8.9 | 10.7 |
| Hydrogenated | 1.2 | | | | | | | |
| Jojoba Oil | | | | | | | | |
| Sorbitol/Sebacic | | 0.5 | | | 2.0 | 2.0 | 1.0 | |
| Acid Copolymer | | | | | | | | |
| Behenate | | | | | | | | |
| Disteardimonium | 0.3 | 0.2 | 0.3 | 0.3 | 0.3 | 0.3 | 0.2 | |
| Hectorite | | | | | | | | |
| Polyhydroxystearic | 0.8 | 1.3 | 1.8 | 1.6 | 1.8 | 2.1 | 1.3 | 3.6 |
| Acid | | | | | | | | |
| Silicons | 17.7 | 17.7 | 17.7 | 17.7 | 17.7 | 17.7 | 17.7 | 17.7 |
| Fatty Compound | 18.3 | 26.1 | 26.0 | 24.1 | 30.4 | 25.5 | 25.6 | 15.0 |
| Surfactant | 2.0 | 4.8 | 4.8 | 4.8 | 4.0 | 4.0 | 4.8 | 0.7 |
| Solvent | 22.1 | 12.0 | 12.0 | 12.1 | 10.6 | 12.0 | 12.0 | 13.5 |
| Active Compound | 1.2 | 1.2 | 1.2 | 1.2 | 1.2 | 1.2 | 1.2 | 0.5 |
| Vitamin | 0.1 | 0.1 | 0.1 | 0.1 | 0.1 | 0.1 | 0.1 | 0.1 |
| Preservative | 0.7 | 0.7 | 0.7 | 0.7 | 0.7 | 0.7 | 0.7 | 0.7 |
| Water | Q.S. | Q.S. | Q.S. | Q.S. | Q.S. | Q.S. | Q.S. | Q.S. |

*All the amounts are by active matters and the quantities of ingredients are expressed by weight percent relative to the total weight of the composition.

### Example 2

### Evaluation

The spray pattern of Inventive and Comparative Examples were evaluated and are the results are presented in Table 2.

TABLE 2
Spray Pattern Results

| | Viscosity | Viscosity | | | |
|---|---|---|---|---|---|
| | at Shear | at Shear | | Fila- | |
| | Rate | rate | Yield | ment | |
| | 0.1 s⁻¹ | 1000 s⁻¹ | Stress | Breakup | Spray |
| Example | (mPa · s) | (mPa · s) | (Pa) | (mSec) | Pattern |
| Inventive Ex. 1 | 2800 | 88.1 | 0.11 | 52 | Good |
| Inventive Ex. 2 | 1160 | 79.9 | 0.17 | 67 | Good |
| Inventive Ex. 3 | 7350 | 95.4 | 0.84 | 76 | Good |
| Inventive Ex. 4 | 1000 | 92.8 | 0.12 | 7 | Good |
| Comparative Ex. 1 | 38390 | 142.4 | 5.4 | 770 | Not good* |
| Comparative Ex. 2 | 15500 | 142.6 | 2.05 | 176 | Not good* |
| Comparative Ex. 3 | 17500 | 119.5 | 1.82 | 110 | Not good* |
| Comparative Ex. 4 | 5280 | 123.3 | 2.1 | 145 | Not good* |

*= not good = bad spray in the figure

### Spray Pattern Evaluation

The spray pattern of inventive and comparative sunscreen formulations was evaluated after filling said formulations into a bag-on-valve (BOV) packaging. The BOV packaging was fitted with a nozzle and actuator system that is typically used for sunscreen compositions containing mineral UV filters and set at a pressure that is typically used for sunscreen compositions containing mineral UV filters. The spray pattern was visualized by holding the can upright at a set distance of around 10-14 cm from a clean, vertically-positioned substrate mounted to the wall. The actuator was depressed once for about 1 second and the resulting spray pattern was assessed visually on overall shape and homogeneity of the distribution of droplets within the resulting shape. A “Not good” quality of spray displayed either inhomogenous droplet size distribution (many large droplets) and/or areas where many droplets were focused. Often the focusing of droplets would create a pattern which had lines or streaks which followed the pattern of the particular channel design within the actuator nozzle. A “Good” quality of spray pattern manifested in an overall pattern that was reasonably circular, and which did not show evidence of focusing of droplets (no discernable aspect of the channels reflected in the pattern).

It should be noted that overall mean droplet size of the spray pattern is controlled by the actuator and nozzle, so the appearance of many large droplets in the spray pattern would be additional evidence of a “Not good” spray pattern.

### Viscosity at Shear Rate 1000 (1/s) Evaluation and Yield Stress Calculation

The viscosity of the inventive and comparative formulations at shear rate of 1000 (1/s) was evaluated using a TA Instruments DHR-2 model hybrid rheometer using 40 mm stainless steel parallel plate geometry. The geometry may also contain cross-hatching to minimize erroneous readings at low shear, but did not affect the viscosity measurements at 1000 (1/s).

Viscosity for each formulation was measured at shear rates ranging from about 0.01 (1/s) up to 1000 (1/s), wherein the resulting viscosity values in the Tables herein represent the viscosity measured at 1000 (1/s).

The yield stress was calculated above data, using Herschel-Bulkley model.

### Filament Breakup Time Evaluation

The filament breakup time (tb) was evaluated under extensional condition using Thermo Scientific™ HAAKE™ CaBER™ 1 Capillary Breakup Extensional Rheometer. Test at 25° C., with plate diameter 6 mm. It is shown in Table 2 that in order to get a good spray pattern, the viscosity of the formula needs to be less than about 100 mPa·s, when the shear rate applied is at about 1000 (1/s) with the yield stress less than about 2 Pa, and the filament breakup time less than about 100 msec.

Those skilled-in-the-art of sunscreen formulation and other sprayable cosmetic formulations would normally understand that sprayability as described in the instant disclosure would be related to the viscosity of the formulation at low shear. In other words, the more fluid the formula, the more likely said formula should be sprayable; however, the inventors discovered in the instant disclosure that a good spray pattern is specifically related to the viscosity at high shear (1000 (1/s) and not viscosity at low shear (for example at 0.1 (1/s) or 1 (1/s). It should have low yield stress, and short filament breakup time to ensure good spray pattern.

Composition exhibits a yield stress less than about 2 Pa, or less than about 1.8 Pa, or less than about 1.5 Pa.

Composition exhibits a viscosity at shear rate of 1000 (1/s) less than 110 mPa·s, or less than about 100 mPa·s, or less than about 90 mPa·s.

Composition exhibits a filament breakup time (tb) less than about 100 msec, or less than about 90 msec, or less than 80 msec.

Therefore, the inventive sunscreen formulations presented in the instant disclosure describe formulations which display a desirable rheological profile to afford a good spray pattern in spray sunscreen compositions that do not require mixing with propellant. This said desirable rheological profile is surprisingly achieved through the control of the rheology modifiers which work in concert with the mineral UV filters and water phase of the inventive formulations.

While the disclosure has been described with reference to described embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is

“At least one,” as used herein, means one or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

The terms “free” and “devoid” indicates that no reliably measurable excluded material is present in the composition, typically 0% by weight, based on the total weight of the composition. The term “essentially free” means that, while it prefers that no excluded material is present in the composition, it is possible to have very small amounts of the excluded material in the composition of the invention, provided that these amounts do not materially affect the advantageous properties of the composition. In particular, “essentially free” means that excluded material can be present in the composition at an amount of less than about 0.1% by weight, based on the total weight of the composition.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

The terms “weight percent” and “wt %” may be used interchangeably and mean percent by weight, based on the total weight of a composition, article or material, except as may be specified with respect to, for example, a phase, or a system that is a component of a composition, article or material. All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1-23. (canceled)

24. A stable sprayable cosmetic composition comprising:
a. at least one mineral UV filter;
b. at least one oil phase rheology modifier; and
c. water;
wherein the cosmetic composition is a water-in-oil composition and,
wherein the composition has a viscosity lower than about 110 mPa·s. at a shear rate of about 1000 (1/s).

25. The cosmetic composition according to claim 24, wherein the viscosity is lower than about 100 mPa·s. at a shear rate of about 1000 (1/s).

26. The cosmetic composition according to claim 24, wherein the viscosity is lower than about 90 mPa·s at a shear rate of about 1000 (1/s).

27. The cosmetic composition according to claim 24, wherein the composition exhibits a yield stress less than about 2 Pa.

28. The cosmetic composition according to claim 24, wherein the yield stress is less than about 1.8 Pa.

29. The cosmetic composition according to claim 24, wherein the yield stress is less than about 1.5 Pa.

30. The cosmetic composition according to claim 24, wherein the composition exhibits a filament breakup time that is less than about 100 ms.

31. The cosmetic composition according to claim 24, wherein the composition exhibits a filament breakup time that is less than about 90 ms.

32. The cosmetic composition according to claim 24, wherein the composition exhibits a filament breakup time that is less than about 80 ms.

33. The cosmetic composition according to claim 24, wherein the at least one mineral UV filter is selected from titanium dioxide, zinc oxide, and mixtures thereof.

34. The cosmetic composition according to claim 24, wherein the at least one mineral UV filter is present from about 5 to about 50 wt. %, based on the total weight of the cosmetic composition.

35. The cosmetic composition according to claim 24, further comprising:
a. One or more organic UV filters.

36. The cosmetic composition according to claim 24, wherein the at least one oil phase rheology modifier is selected from hydrogenated jojoba oil, disteardimonium hectorite, polyhydroxystearic acid, sorbitol/sebacic acid copolymer behenate, and mixtures thereof.

37. The cosmetic composition according to claim 24, wherein the at least one oil phase rheology modifier is present from about 0.01 to about 5 wt. %, based on the total weight of the cosmetic composition.

38. The cosmetic composition according to claim 37, wherein the at least one oil phase rheology modifier is present from about 0.1 to about 3 wt. %, based on the total weight of the cosmetic composition.

39. The cosmetic composition according to claim 24, where when the composition is sprayed the distribution of droplets is homogenous.

40. The cosmetic composition according to claim 24, wherein the composition is aerosolized without requiring mixing with propellant.

41. The cosmetic composition according to claim 24, wherein the viscosity is less than about 100 mPa·s at a shear rate of about 1000 (1/s).

42. The cosmetic composition according to claim 24, wherein said composition is a sprayable composition.

43. A stable sprayable water-in-oil cosmetic composition comprising:
a. from about 5 to about 50 wt. % of at least one mineral UV filter;
b. from about 0.1 to about 5 wt. % of at least one oil phase rheology modifier;
c. from about 10 to about 30 wt. % of water; and
wherein the cosmetic composition is a water-in-oil composition and,
wherein the composition has a viscosity lower than about 100 mPa·s at a shear rate of about 1000 (1/s);
wherein all percentages by weight are based on the total weight of the cosmetic composition.

44. A method for protecting skin from UV radiation comprising applying an effective amount of the sunscreen composition of claim 24 to the skin.
